(19) United States
(12) Patent Application Publication
(10) Pub. No.: US 2013/0323714 A1
(43) Pub. Date: Dec. 5, 2013

(54) SYSTEM AND METHOD FOR MICRO-AERATION BASED FERMENTATION

(71) Applicants: Alan T. Cheng, Naperville, IL (US); Ying Zhou, Naperville, IL (US); Paul W. Belanger, Clarence Center, NY (US); Jennifer G. Bugayong, Kenmore, NY (US)

(72) Inventors: Alan T. Cheng, Naperville, IL (US); Ying Zhou, Naperville, IL (US); Paul W. Belanger, Clarence Center, NY (US); Jennifer G. Bugayong, Kenmore, NY (US)

(21) Appl. No.: 13/908,462

(22) Filed: Jun. 3, 2013

Related U.S. Application Data
(60) Provisional application No. 61/655,117, filed on Jun. 4, 2012.

(51) Int. Cl. C12M 1/00

(52) U.S. Cl. CPC C12M 29/24
USPC 435/3; 435/300.1; 435/287.1; 435/286.6

(57) ABSTRACT

A method and apparatus for micro-aeration of large scale fermentation systems is provided. The micro-aeration system includes a fermentation reactor, a sparging apparatus, and a micro-aeration gas mixture delivered to the fermentation reactor via the sparging apparatus. The micro-aeration gas mixture is a very low oxygen concentration mixture comprising an oxygen containing gas and an inert carrier gas that is preferably recycled through the fermentation reactor. The inert carrier gas is preferably nitrogen whereas the oxygen containing gas is oxygen or and is introduced to the fermentation reactor at a minimum superficial velocity of about 0.02 m/sec to produce a uniform dispersion of the oxygen/air throughout the fermentation broth while concurrently mixing the entire fermentation broth. The micro-aeration method and apparatus further comprises a controller operatively coupled to one or more control valves for regulating the micro-aeration conditions in the fermentation reactor.

### CROSS REFERENCE TO RELATED APPLICATIONS

This application claims priority to and the benefit of U.S. provisional patent application Ser. No. 61/655,117 filed on Jun. 4, 2012, the disclosure of which is incorporated by reference herein.

### FIELD OF THE INVENTION

The present invention broadly relates to fermentation processes, and more particularly, to devices, methods, and systems for providing, via micro-aeration, a controlled rate and concentration of highly diluted air or oxygen into and through a fermenter using an inert carrier gas, preferably nitrogen. By controlling the bulk flow rate and concentration of gas flows into the fermenter, the build-up and concentration of carbon dioxide can also be properly controlled. The primary purpose of providing these systems is for optimization of yields of biologically derived fermentation of microbes that in turn produce desirable chemical commodities such as alcohols and organic acids on a large industrial scale. The ability to use a large gas volume of an inert carrier provides for ease of control and for greater dispersion by providing the diluted air or oxygen to be homogeneously spread throughout any fermentation broth.

### BACKGROUND

Depending on the availability of oxygen, microbes such as e-coli, yeast, fungus, etc. have different metabolic pathways and/or enzymes for the consumption of different kinds of nutrients or quantities of the same raw materials leading to production of different kinds of products or yields of the same products. Anaerobic fermentation is a process widely used to produce desirable chemical commodities such as food grade acids and alcohols, because it is generally viewed as the most efficient form of fermentation used in the consumption of carbon (i.e. sugar) sources. However, such microbes are generally less robust during anaerobic conditions in the consumption of wide ranges of complex nutrients and less tolerant of toxic concentration levels of the products or byproducts being produced. Certain of these microbes used in fermentation processes favor aerobic fermentation due to their high cell growth rate and production capacity. It is known, however, that these same microbes, under high oxygen tension, waste more nutrients during cell growth and may actually consume the desirable products that are being produced, thus resulting in product yield losses. Micro-aeration is a technique that allows for fermentation to proceed somewhere between anaerobic and aerobic fermentation with very low levels of oxygen or under oxygen limiting conditions present. Metabolic pathways for the microbes prompted by micro-aeration can generate higher yields than either pure aerobic or pure anaerobic processes.

Practicing micro-aeration techniques in commercial scale or large scale fermenters with commercial success however, is very difficult. Large scale anaerobic fermenters are used to produce products such as biofuels (e.g. bioethanol and biobutanol) can easily exceed 1,000,000 liters in volume. The amount of oxygen or air required for the micro-aeration can be up to 50 or 100 times smaller than the amount of oxygen or air typically needed for aerobic fermentation. Monitoring, controlling and dispersing the small amount of oxygen or air required in the fermenter are major issues in applying the micro-aeration technique to commercial scale or large scale fermentation operations. Anaerobic fermenters typically have no air spargers and no agitators. To adopt micro-aerobic for an anaerobic fermenter, one would normally add an open pipe or sparger to provide the needed amount of air or oxygen. However, sparging gas into a large fermenter without proper dispersing or bulk mixing causes some microbes to receive excess oxygen while others may be totally depleted of oxygen, even when the average oxygen uptake throughout the fermenter appears to be acceptable.

Laboratory studies of micro-aeration techniques generally report results showing fermentation product yield improvements at specific dissolved oxygen (DO) levels. In order to measure DO levels during micro-aeration, the use of DO sensors or probes to measure and maintain the DO levels in the fermentation broth is common In practice, however, these laboratory conditions that produce the improved product yields cannot be easily duplicated or replicated in commercial or large scale fermenters. This is because DO levels of the fermentation broth in commercial or large scale fermenters change significantly depending on the spatial location in the fermentation vessel or reactor from the oxygen or air sparger and/or any mechanical agitators in the vessel, and more particularly, change significantly along the vertical height of the fermenter.

Several of the reasons that the DO levels vary significantly depending on the spatial location in the fermentation vessel or reactor and more particularly along the vertical height of the fermenter include: (i) as oxygen or air bubbles rise in the vessel, the diameter of the bubbles and oxygen concentration dynamics change, resulting in changes of the mass transfer driving force; and (ii) since oxygen is constantly being consumed by the microbes, oxygen concentration gradients appear as one moves further away from the oxygen or air sparger, generally resulting in higher DO levels proximate the spargers or oxygen source (e.g. near the bottom of the fermentation broth) and lower DO levels further away from the spargers (e.g. near the top of the fermentation broth). As a result, many prior art fermentation systems are overly concerned with sparger configuration and location as well as the location and operation of mechanical agitators in the fermentation vessel or reactor. Notwithstanding these efforts, in many commercial scale or large scale fermenters there remains certain spatial locations within the fermenter that are completely starved of oxygen and other areas where excessive oxygen is consumed.

The published patent application number WO 2012/018699 to Myriant describes a method of producing bio-succinic acid using a micro-aeration technique. Carbon dioxide is an important nutrient for the production of succinic acid as the microbes incorporate the carbon source from inorganic bicarbonate and carbon dioxide into the building block of bio-succinic acid. To run a fermentation process under specific micro-aeration conditions, small amounts of air (i.e. <1% by volume) is added to the carbon dioxide feed stream to generate a gas mixture. However, such process is not suitable for many fermentation processes where excessive amounts of carbon dioxide have adverse effects on the process.

The Myriant publication further discloses that by providing a minimal amount of oxygen during the production phase, the yield and productivity of the succinic acid is improved. With the microaerobic condition during the production phase, there is a better utilization of organic carbon present in the medium as opposed to utilization of only 80% of the organic carbon under strict anaerobic conditions during the production phase. The enhanced carbon utilization during the microaerobic production phase is further accompanied by a noticeable increase in the yield and productivity of the succinic acid.

The micro-aerobic condition allegedly achieved in the Myriant reference was achieved by means of mixing a small amount of air with the carbon dioxide nutrient. The dissolved oxygen (DO) level in the fermentation broth is monitored using an oxygen electrode or any other suitable oxygen sensing device and the flow rate of the gas mix is adjusted to assure that the level of oxygen in the fermentation broth is maintained at a constant level. It is important to note that the Myriant reference involves fermentation of succinic acid and the carbon dioxide gas is a needed nutrient in this process. As a result, the gas flow rate sparged into the fermentation vessel must be slow enough so that the majority of carbon dioxide will be consumed as a nutrient.

This Myriant publication states that fermentation reaction vessels of any suitable, known type may be employed in performing the micro-aeration based fermentation process, including packed bed reactors, continuous stirred tank reactors, rotating biological contact reactors, sequencing batch reactors and fluidized bed reactors. The size of the fermentation reaction vessels is disclosed in the Myriant reference is the range of 3 L to 400,000 L and the fermentation can be carried out in a continuous process, a batch mode or a fed-batch mode, with the fed-batch mode preferred.

Because the carbon dioxide gas is introduced slowly into the fermentation broth and rapidly consumed as a nutrient in the Myriant process, the use of the Myriant micro-aeration process in large scale fermenters will still result in unbalanced DO levels throughout the fermeter with higher DO levels realized at locations proximate the spargers and lower DO levels realized at locations further away from the spargers. To achieve relatively uniform DO levels throughout a commercial scale fermenter using the Myriant micro-aeration process, one would require a complex sparger network and preferably use of mechanical agitators. This is primarily a result of the slow gas flow rates and rapid consumption of the carbon dioxide gas in the Myriant process which fails to provide proper dispersion or bulk mixing of the small amounts of oxygen throughout the fermentation broth.

What is needed therefore are micro-aeration systems and methods for solving problems of inconsistencies in gas dispersion, mass transfer limitations, poor bulk mixing, control and operating issues in large or commercial scale fermentation systems.

### SUMMARY OF THE INVENTION

The present invention may be characterized as a micro-aeration based fermentation system comprising: (i) a fermentation reactor; (ii) a sparging apparatus disposed in the fermentation reactor; (iii) a micro-aeration gas mixture delivered to the fermentation reactor via the sparging apparatus, the micro-aeration gas mixture comprising an oxygen containing gas and an inert carrier gas; and (iv) an off-gas recycle loop configured to recycle off-gases exiting the fermentation reactor back to the sparger apparatus. The oxygen concentration in the micro-aeration gas mixture is less than or equal to 20%, and the micro-aeration gas mixture is delivered to the fermentation reactor at a minimum superficial velocity of 0.02 m/sec to mix the fermentation broth within the fermentation reactor and disperse the oxygen throughout the fermentation broth.

The inert carrier gas is preferably nitrogen whereas the oxygen containing gas is oxygen, air or a mixture thereof and the sparging apparatus is for delivering the gas flows to the fermentation broth is preferably disposed proximate the bottom of the fermentation reactor. The fermentation vessel or reactor is preferably configured without mechanical agitators and the reactor has a height to diameter ratio of at least about 3 to 1 such that the reactor configuration provides an uplifting action of the inert carrier gas ensuring heterogeneous flow of the oxygen containing gas throughout the reactor.

The micro-aeration system also includes one or more control valves responsive to a central programmable logic controller (PLC) for regulating the flows of the inert carrier gas and oxygen containing gas delivered to the fermentation reactor. More specifically, the volume of the oxygen containing gas and inert carrier gas delivered to the fermentation reactor are controlled or regulated in response to a respiratory quotient (RQ); an oxygen transfer rate (OTR), an oxygen uptake rate (OUR), and/or a carbon dioxide evolution rate (CER) associated with the fermentation broth, as calculated using gas analyzers to determine the oxygen and carbon dioxide concentrations of the off-gas in the recycle loop as well as one or more flow meters, dissolved oxygen (DO) probes, cell density meters, and other probes or sensors.

The invention may also be characterized as a method of micro-aeration of a fermentation broth comprising the steps of: (i) mixing an inert carrier gas flow with an oxygen containing gas flow and an off-gas recycle flow to produce a micro-aeration gas mixture having an oxygen concentration of less than about 20% by volume; (ii) sparging the micro-aeration gas mixture to the fermentation broth in a fermentation reactor via a sparging apparatus at a minimum superficial velocity of 0.02 m/sec to mix the fermentation broth within the fermentation reactor and uniformly disperse the oxygen containing gas throughout the fermentation broth; (iii) recycling some or all of the off-gases produced by the fermentation broth in the fermentation reactor back to the sparger apparatus via an off-gas recycle loop; (iv) measuring one or more of the gas flows selected from the group consisting of the off-gas recycling flow, the inert carrier gas flow, the oxygen containing gas flow, and the micro-aeration gas flow; (v) ascertaining selected parameters representing the physical and chemical characteristics of the fermentation broth in the fermentation reactor and selected gas concentrations in the off-gas recycle loop; and (vi) controlling the flows of the inert carrier gas and the oxygen containing gas in response to the selected parameters.

It should be appreciated that all combinations of the foregoing concepts and additional concepts discussed in greater detail below are contemplated as being part of the inventive subject matter disclosed herein. In particular, all combinations of claimed subject matter appearing at the end of this disclosure are contemplated as being part of the inventive subject matter disclosed herein.

The foregoing and other aspects, embodiments, and features of the present teachings can be more fully understood from the following detailed description of the preferred embodiments taken in conjunction with the accompanying drawings.

### BRIEF DESCRIPTION OF THE DRAWINGS

While the specification concludes with claims distinctly pointing out the subject matter that Applicants regard as their invention, the invention will be better understood when taken in connection with the accompanying drawings in which:

FIG. 1 is a schematic view of an embodiment of a micro-aeration based fermenter system in accordance with the present invention; and

FIG. 2 is a schematic view of the micro-aeration based fermenter system shown in FIG. 1 further illustrating a preferred control system and scheme;

FIG. 3 is a schematic view of an alternate embodiment of a micro-aeration based fermenter system in accordance with the present invention;

FIG. 4A is a graph depicting the comparative results of BDO yield versus average OUR in BDO fermentation without RQ control;

FIG. 4B is a graph depicting the comparative results of BDO volumetric productivity versus average OUR in BDO fermentation without RQ control;

FIG. 4C is a graph depicting the comparative results of BDO yield versus RQ in BDO fermentation with RQ control; and

FIG. 4D is a graph depicting the comparative results of BDO volumetric productivity versus RQ in BDO fermentation with RQ control.

### DETAILED DESCRIPTION

The present system is a micro-aeration system that provides the ability to disperse small amounts of an oxygen containing gas for micro-aeration into a large volume fermentation reactor with a microbe containing fermentation broth. These microbes (e.g. e-coli, yeast, fungus, etc.) possess different metabolic pathways for consumption of different kinds of nutrients or quantities of raw materials and produce different kinds of products or quantities of fermentation products. Anaerobic fermentation is widely used because it is typically viewed as the most efficient in use of the carbon (i.e. sugar) source to produce desirable chemical commodities, such as acids and alcohols.

For purposes of this disclosure, micro-aeration conditions may be generally defined as having the dissolved oxygen (DO) level between about 0.01% to 20.0% of the air saturation level or preferably between about 0.1% to 8.0% of air saturation level, during the production phase of the fermentation. However, it is difficult to measure accurately the low DO level under fermentation conditions and an extreme active microbe might drive the DO level to very low even it is physiologically consuming a large volume of oxygen under aerobic conditions. Therefore, micro-aeration in this invention is better defined as having oxygen uptake rate between about 0.01 mmoles O₂/L-hr to 50 mmoles O₂/L-hr or more preferably between about 0.1 mmoles O₂/L-hr to 10.0 mmoles O₂/L-hr of oxygen being consumed during the production phase of the fermentation.

The presently disclosed micro-aeration based fermentation system provides very large and excessive volumes of an inert carrier gas as a diluent to the fermentation vessel or reactor to uniformly disperse the small amounts of gaseous oxygen within the fermentation vessel or reactor. Preferably, the carrier gas is not a required nutrient for the fermentation broth but instead is an inert gas that, when used in excess, creates a large gas flow in order to disperse the oxygen into the fermentation broth and assist with mixing of the entire fermentation broth. The inert carrier gas is preferably nitrogen, other inert gases, or mixtures thereof. However, in some narrow fermentation applications, a nutrient containing carrier gas can be used instead of or together with the inert carrier gas.

By using an inert carrier gas instead of a reactive or nutrient containing carrier gas, the inert carrier gas will not be consumed by the microbes as it moves up the fermentation vessel or reactor. The inert carrier gas therefore allows for continuous rising inert gas bubbles to produce the mechanical work of mixing from the bottom of the fermenter to the top of the fermenter and ensures vigorous mixing of the entire tank. The excessive flow of the inert carrier gas preferably has a minimum superficial velocity of about 0.02 m/sec and more preferably about 0.05 m/sec or higher and thus transforms the fermentation vessel or reactor into a pseudo bubbling column similar to a nitrogen bubbling column. This thorough and vigorous mixing is critical to bring optimum level of nutrient and dissolved oxygen to every microbe in the fermentation broth in a commercial scale fermenter. This vigorous mixing also tends to avoid the segregation of the fermentation broth into regions or zones of high levels and low levels of DO.

The excess volume and flow of the inert carrier gas not only provides the requisite agitation or mixing of the fermentation broth in an agitator free fermentation reactor but also aids in stripping of unwanted gases and volatiles, including excess carbon dioxide that would otherwise be harmful for microbes in the fermentation broth.

For a typical nitrogen bubbling column with a 5 to 1 height to diameter ratio, the nitrogen flow rate required to form heterogeneous bubbling flow required for effective bulk mixing is at least 900 ft³/min, with the heterogeneous bubbling flow comprising a mixture of small and large gas bubble sizes. Such high gas flow rates moving through the fermentation reactor of the present micro-aeration based fermentation system and method are significantly higher than the gas flow rates described in prior art micro-aeration systems. As indicated above, the significantly higher carrier gas volume and flow rates allow the desired amount of oxygen to be uniformly dispersed throughout the fermentation reactor to create an optimum micro-aeration process.

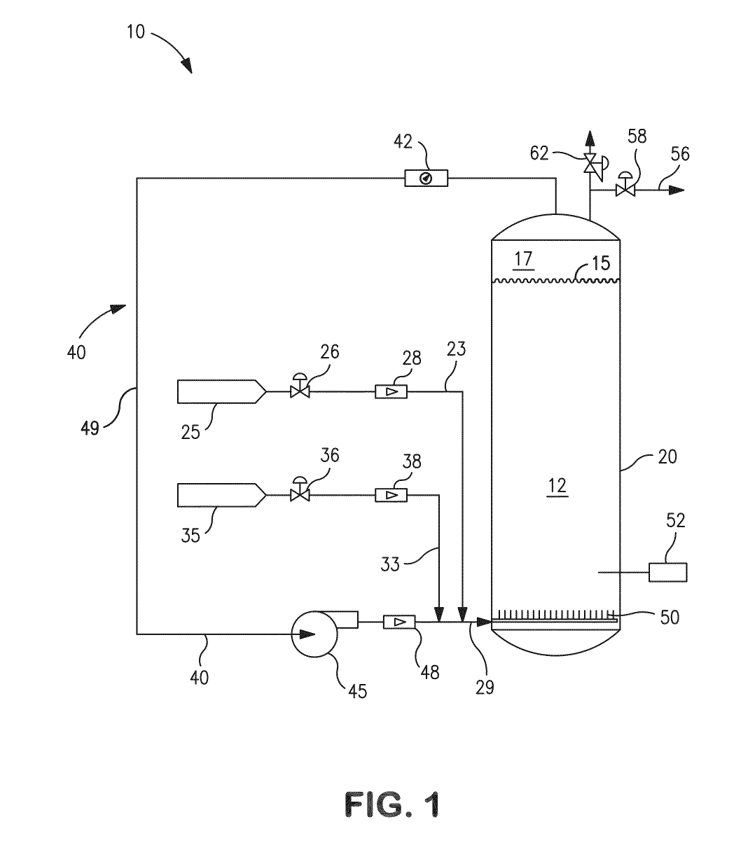

Turning now to the drawings, FIG. 1 is a schematic view of an embodiment of a micro-aeration based fermenter system 10. As seen therein, the micro-aeration based fermenter system 10 comprises a fermentation vessel or reactor 20, a sparger apparatus 50; and a micro-aeration gas mixture 29 delivered to the fermentation reactor 20 via the sparging apparatus 50, the micro-aeration gas mixture 29 comprising an oxygen containing gas 35 and an inert carrier gas 25. In this preferred embodiment, the total oxygen concentration in the micro-aeration gas mixture is less than or equal to 20%, and wherein the micro-aeration gas mixture 29 is delivered to the fermentation reactor 20 at a minimum superficial velocity of about 0.02 m/sec to provide mixing of the fermentation broth 12 within the fermentation reactor 20 and uniformly disperse the oxygen.

FIG. 1 also shows a supply of nitrogen gas 25 or other appropriate inert carrier gas and a source of oxygen containing gas 35. The nitrogen gas 25 is preferably supplied via a liquid nitrogen storage tank, membrane nitrogen, pressure swing adsorption (PSA) system or on-site cryogenic air separation plant. The source of nitrogen gas 25 is supplied via a first gas line 23 through a control valve 26 and flow meter 28 to the sparger apparatus 50. The source of oxygen containing gas 35 is oxygen or air and is supplied via a second gas line 33 and also provided to the sparger apparatus 50 after it is combined with the nitrogen carrier gas 25. A second control valve 36 and second flow meter 38 are used to monitor and control the amount of oxygen or air 25 being mixed from the second gas line 33 with the nitrogen or inert carrier gas in the first gas line 23.

The combined flow is the micro-aeration gas mixture 29 introduced to the fermentation reactor 20 via the sparger apparatus 50 where a portion of the oxygen is consumed by the microbes within the fermentation broth 12. The fermentation broth 12 in the fermentation reactor 20 is continually monitored using one or more sensors such as an optical density meter 52 (i.e. to measure cell density), dissolved oxygen (DO) probes, etc. Preferably, the fermenter reactor 20 has a height to diameter ratio of about 3 to 1 or greater and the sparging apparatus 50 is disposed proximate the bottom of the fermenter reactor 20 so that the uplifting action of the rising heterogeneous flow of the micro-aeration gas mixture 29 is realized which provides both effective mixing of the fermentation broth 12 within fermentation reactor 20 and uniform dispersion of the oxygen throughout the entire volume of the fermentation broth 12. As the rising heterogeneous flow of the micro-aeration gas mixture 29 reaches the top surface 15 of the fermentation broth 12, an off-gas is released to the headspace 17 and exits the fermentation reactor 20. As described in more detail below, the off-gases from the headspace 17 may be purged 56 from the fermentation reactor 20 via purge control valve 58 and/or vented via valve 62. The rate of off-gas purge is dictated by build-up in carbon dioxide concentration in the off-gas and pH changes in the fermentation broth or a combination of both. In addition, some or all of the off-gases from the headspace 17 of the fermentation reactor 20 may be recycled back to the sparger apparatus 50 via recycle loop 40.

As seen in FIG. 1, a gas analyzer 42 is preferably disposed in recycle loop 40. The gas analyzer 42 is configured to ascertain the levels of oxygen, carbon dioxide and other gas components of the recycled off-gas from which parameters such as respiratory quotient (RQ), oxygen transfer rate (OTR), oxygen uptake rate (OUR) and carbon dioxide evolution rate (CER) are calculated and used to control the micro-aeration process. While use of OTR and OUR parameters are common in the control of oxygen based fermentation systems, the use of RQ and CER parameters are less common. The respiratory quotient (RQ) is the calculated ratio CER/OUR where the CER is an expression of the rate at which carbon dioxide is produced by the fermentation process.

In addition to the gas analyzer 42, the recycle loop also preferably includes a gas blower/compressor 45 and flow meter 48. The gas compressor 45 is configured to forcibly recirculate the off-gases exiting from the headspace 17 of the fermentation reactor 20 back to the sparger apparatus 50 disposed proximate the bottom of the fermentation reactor 20. The flow meter 48 is configured to measure the flow rate of the recycled off-gas 49 in the recycle loop which is used to ensure the minimum superficial velocity of the micro-aeration gas is maintained and used to adjust the amount of supplemental or make-up carrier gas and make-up oxygen needed to maintain the micro-aeration conditions. Biological filters may also be installed in operative association with the nitrogen or inert carrier gas 25 in the first gas line 23 as well as with the oxygen/air source 35 in the second gas line 33 to avoid microbe contaminations. Biological filters may also be disposed in the recycle loop 40.

The sparger apparatus 50 is preferably disposed proximate the bottom of the fermenter 20 so as to facilitate uniform dispersion of the small amounts of gaseous oxygen or air within the fermentation vessel or reactor. Typical nitrogen bubbling column spargers or horizontal pipe spargers having a plurality of drilled holes oriented along the lengths of the pipe that are capable of achieving a minimum superficial velocity of about 0.02 m/sec or higher have been shown to be effective to achieve the desired micro-aeration within the fermenter. Alternatively, metallic membrane spargers can also be used in lieu of the horizontal pipe sparger network with drilled holes.

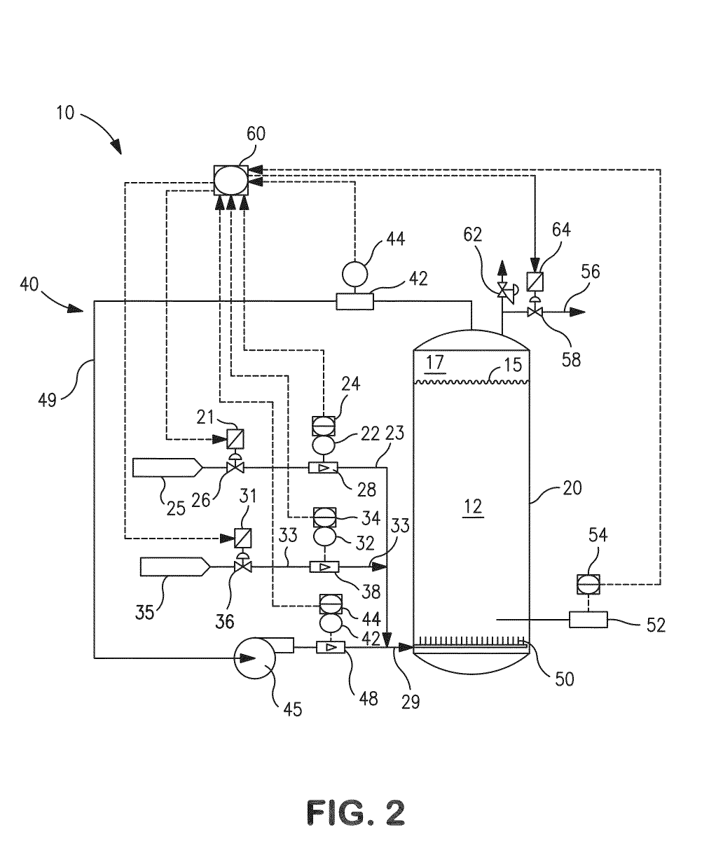

Turning now to FIG. 2, there is shown the schematic view of the micro-aeration based fermenter system of FIG. 1 further illustrating a preferred control system and scheme. As seen therein, the advanced control system comprises a central programmable logic controller (PLC) 60 operatively coupled via proportional-integral (P/I) controllers 21, 31, and 64 to control valve 26, control valve 36; and purge control valve 58, respectively. Specifically, control signals from the PLC 60 are passed to the oxygen/air control valve 36 and carrier gas control valve 26 to regulate the oxygen concentration and carrier gas concentration in the micro-aeration gas mixture 29 delivered to the fermentation reactor 20 via the sparger apparatus 50.

Inputs to the PLC 60 include measured values of the carrier gas flow via flow indicator 22 and flow transmitter 24; measured values of the oxygen containing gas flow via flow indicator 32 and flow transmitter 34; and measured values of the recycle gas flow via flow indicator 42 and flow transmitter 44. Additional critical inputs to the PLC 60 further include the cell density in the fermentation broth 12 as measured by the optical density meter 52 and input to the PLC 60 via cell density transmitter 54 as well as outputs from the gas analyzer 42 which are sent to the PLC 60 via indicator/transmitter 44. Although not shown, other sensors and probes may also be incorporated into the present advanced control scheme. For example, a pressure transducer/transmitter may be used to provide an input signal to the PLC 60 so as to maintain constant pressure control in the system and balance the off-gas purge and make-up gas additions. Additional sensors such as dissolved oxygen probes and temperature sensors may also desired to assist in controlling the overall micro-aeration based fermentation process. Precisely controlling the flow rates in response to the measured parameters, and more particularly to a set of calculated parameters is critical and the PLC 60 is configured to calculate the set points required to control the gas flows and purge flow throughout the fermentation process.

The concentration of oxygen provided to the fermentation broth 12 and the carbon dioxide in the fermentation broth 12 are regulated or controlled by measuring the off-gas oxygen concentration and carbon dioxide concentration so that key parameters such as RQ, OTR, OUR, and CER can be properly ascertained. It is important to note that such key parameters are typically not constant during the fermentation process as the cell density continually changes throughout the fermentation cycle and the oxygen demand will vary during the fermentation process even in micro-aeration conditions when the amount of oxygen available for individual microbes is limited. Therefore, cell density is preferably measured during the fermentation process using an on-line optical density meter 52 to determine the change in cell densities. High cell densities typically increase the demand for more oxygen. The optical density measurements are transmitted to the PLC 60 via cell density transmitter 54 to determine if any oxygen adjustment is needed. The PLC 60 sends set point signals to electro-pneumatic control valves 26 and 36 to regulate the nitrogen and air/oxygen make-up flows.

Since the micro-aeration gas mixture 29 is capable of stripping out toxic or inhibiting volatile compounds including carbon dioxide, part of the recirculating nitrogen gas stream may be purged to avoid buildup of those volatile compounds. Supplemental or make-up nitrogen is then added into the recirculating gas stream to maintain the total flow rate and minimum superficial velocity. In the illustrated embodiments, the PLC 60 controls how much off-gas must be purged and how much make-up nitrogen and air/oxygen is required to compensate for the off-gas purge. The off-gas purge rate or frequency should be increased if the volatile compounds are found to cause inhibiting or toxic effects on the fermentation broth 12. For example, in anaerobic fermentation based ethanol production processes, removing the carbon dioxide in an efficient and optimal manner improves and/or accelerates the ethanol production rate substantially.

Instead of using the DO levels as the feedback control parameter as typically done in many prior art micro-aeration systems, the present embodiments use gas phase oxygen concentration and/or carbon dioxide concentration as control points. This is because the typical DO probes become highly unreliable when DO levels approach zero as is the case in some micro-aeration conditions. It is possible that the noise associated with data instrumentation in an operating fermenter can be higher than the set point or measured value of a micro-aeration process. Using gas phase oxygen for the control point is one way to overcome this limitation.

The oxygen transfer rate (OTR), oxygen uptake rate (OUR) or carbon dioxide evolution rate (CER) and respiratory quotient (RQ) are all potentially calculated parameters to be used for feedback control for the present micro-aeration based fermentation system and method. Such parameters are calculated from material balances based on the measured gas flow rates and concentration of oxygen and carbon dioxide as measured by the gas analyzers.

### EXAMPLE

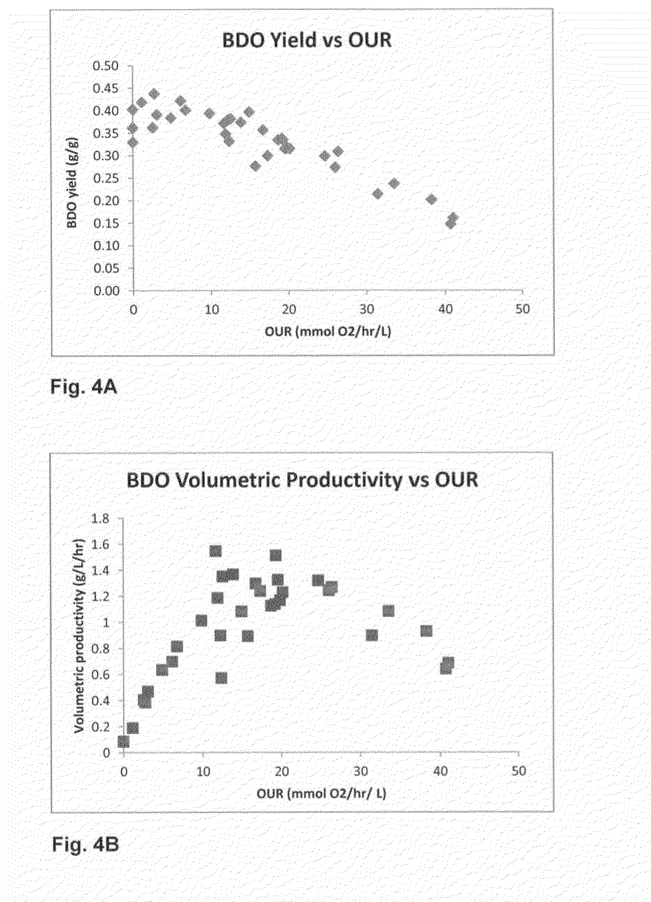

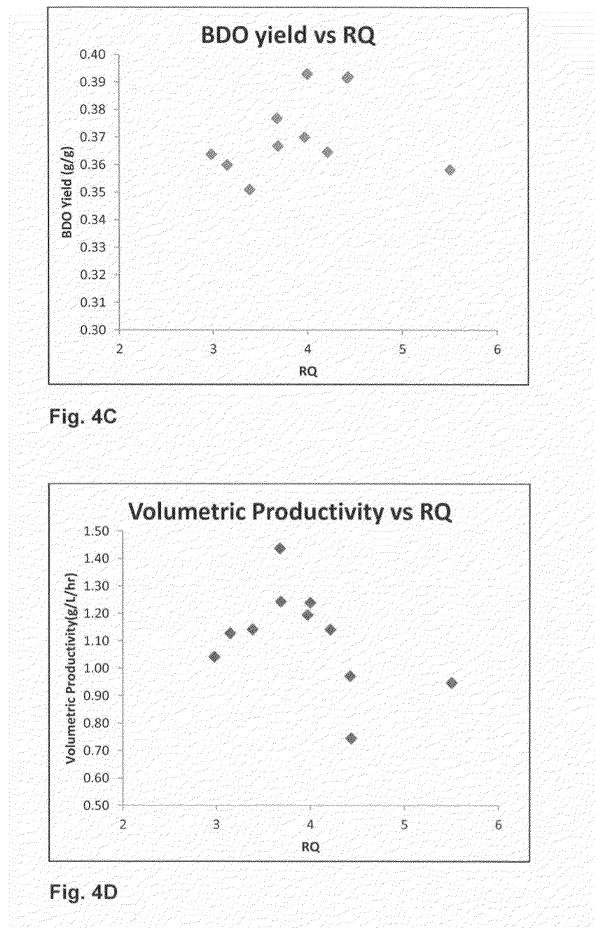

With reference to FIGS. 4A, 4B, 4C, and 4D, the following example shows the benefit of using respiratory quotient (RQ) as the preferred control parameter for micro-aeration based fermentation. As discussed above, RQ is defined as the molar ratio of the carbon dioxide evolution rate (CER) to the oxygen uptake rate (OUR). The OUR and CER were determined by measuring the gas flow rates and the concentrations of carbon dioxide and oxygen in the recycled off-gas. Under pseudo-steady-state and oxygen-limited conditions, the OTR can be calculated from the OUR. The comparison of 2, 3-BDO fermentation with RQ control and without RQ control was conducted using a 5 L fermenter employing the micro-aeration scheme as generally illustrated in FIG. 2. The control of OUR and RQ was realized by adjusting the aeration rate of the flow to the sparger.

When control of the micro-aeration based fermentation process is based simply on OUR (i.e. without RQ control), the maximum BDO yield (g/g) from the fermentation process occurred when the average OUR was lower than about 7.0 mmol O₂/hr/L (See FIG. 4A) whereas the maximum volumetric productivity (g/L/hr) occurred when the average OUR was between about 12.0 to 20.0 mmol O₂/hr/L (See FIG. 4B). As a result, there is a larger trade-off between maximum BDO yield and maximum volumetric productivity when the micro-aeration control is based simply on the OUR.

On the other hand, when control of the micro-aeration based fermentation process is based on RQ, the maximum BDO yield (g/g) from the fermentation process occurred when the RQ was between about 4.0 and 4.5 (See FIG. 4C) whereas the maximum volumetric productivity (g/L/hr) occurred when the RQ was between about 3.5 and 4.0 (See FIG. 4D). Thus, there was less need trade-off between maximum BDO yield and maximum volumetric productivity when the micro-aeration control is based on RQ, and in particular when the RQ is controlled at about 4.0, the overlapping regions of maximum BDO yield and maximum volumetric productivity.

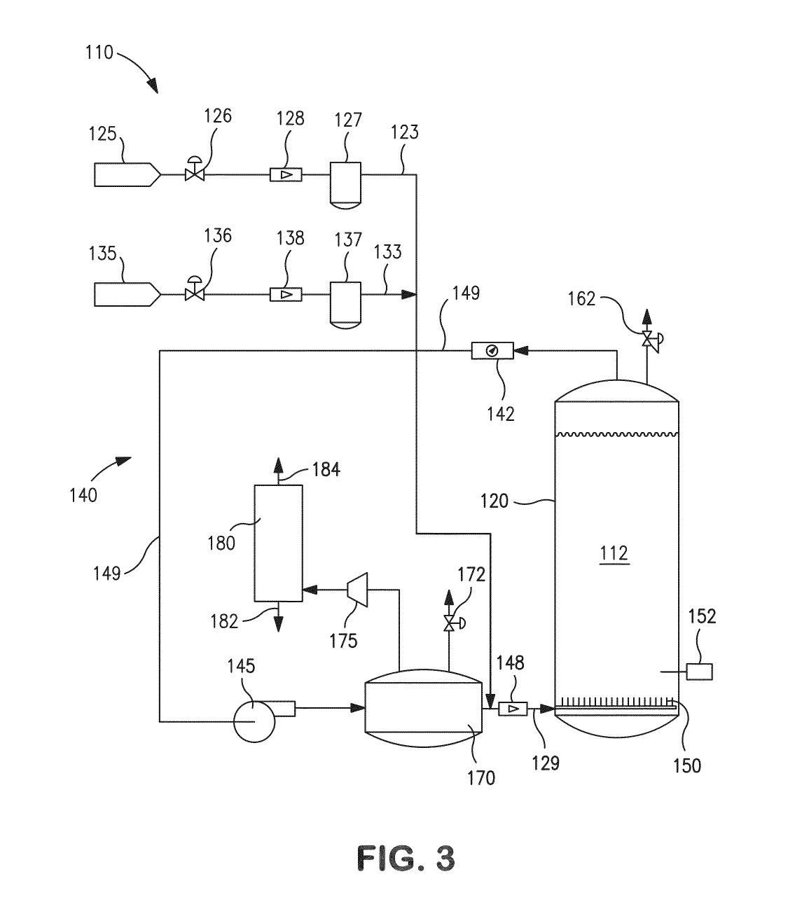

Turning now to FIG. 3, there is shown a schematic view of an alternate embodiment of a micro-aeration based fermenter system. Similar to the embodiment described above with reference to FIG. 1 and FIG. 2, the micro-aeration based fermenter system 110 comprises a fermentation vessel or reactor 120, a sparger apparatus 150; and a micro-aeration gas mixture 129 delivered to the fermentation reactor 120 via the sparging apparatus 150. As with the earlier embodiments, the micro-aeration gas mixture 129 comprising an oxygen containing gas 125 and an inert carrier gas 135 with the total oxygen concentration in the micro-aeration gas mixture being less than or equal to 20% and wherein the micro-aeration gas mixture 129 is delivered to the fermentation reactor 120 at a minimum superficial velocity of about 0.02 m/sec to provide both micro-aeration and mixing of the fermentation broth 12.

FIG. 3 also shows the supply of nitrogen gas 125 or other appropriate inert carrier gas and a source of oxygen containing gas 135 or air. The source of nitrogen gas 125 is supplied via a first gas line 123 through a control valve 126 and flow meter 128 and directed to the sparger apparatus 150. The source of oxygen containing gas 135 or air is supplied via a second gas line 133 and combined with the nitrogen carrier gas 125. A second control valve 136 and second flow meter 138 are used to monitor and control the amount of oxygen or air being mixed with the nitrogen or inert carrier gas. The combined flow is the micro-aeration gas mixture 129 introduced to the fermentation reactor 120 via the sparger apparatus 150 where a portion of the oxygen is consumed by the microbes within the fermentation broth 112. The fermentation broth 112 in the fermentation reactor 120 is continually monitored using one or more sensors such as an optical density meter 152, DO probes, temperature sensors, pH sensors and the like. Preferably, the fermenter reactor 120 has a height to diameter ratio of at least about 3 to 1 with the sparging apparatus 150 disposed proximate the bottom of the fermenter reactor 120. This arrangement ensures the uplifting action of the rising micro-aeration gas mixture 129 is realized which, in turn, provides both effective mixing of the fermentation broth 112 within fermentation reactor 120 and uniform dispersion of the oxygen throughout the entire volume of the fermentation broth 112. As the rising heterogeneous flow of the micro-aeration gas mixture 129 reaches the top surface 115 of the fermentation broth 112, an off-gas is released to the headspace 117 and exits the fermentation reactor 120. In this embodiment, the off-gases from the headspace 117 may be vented via valve 162, or more preferably, purified or cleansed of unwanted volatiles and carbon dioxide and recycled back to the fermentation vessel via recycle loop 140.

Similar to the earlier embodiments, a gas analyzer 142 is operatively disposed in the recycle loop 40. The gas analyzer 142 is configured to ascertain the levels of oxygen, carbon dioxide and other gas components of the recycled off-gas 149 from which parameters such as RQ, OTR, OUR and CER are calculated and used to control the micro-aeration process as generally described above with reference to FIG. 2.

A gas compressor 145 is also disposed in the recycle loop 140 and is configured to forcibly recirculate the off-gases exiting from the headspace 117 of the fermentation reactor 120 back to the sparger apparatus 150. Flow meter 148 is configured to measure the gas flow rate in the recycle loop 140 which is used to adjust the amount of supplemental or make-up carrier gas and make-up oxygen needed to maintain the minimum superficial velocity of the micro-aeration gas stream 129.

A first biological filter 127 is preferably installed in operative association with the nitrogen or inert carrier gas 125 in the first gas line 123 and another biological filter 137 is preferably installed in operative association with the oxygen/air source 135 in the second gas line 133 to avoid microbe contaminations. Additional biological filters may also be disposed in the recycle loop 40.

What differs in this embodiment from the embodiment shown and described with reference to FIGS. 1 and 2, is the presence of a carbon dioxide stripping process applied to the recycled off-gas 194. The carbon dioxide stripping subsystem 200 comprises a carbon dioxide variable pressure swing adsorption (VPSA) system 170 disposed in the recycle loop 140 and operatively adsorbing carbon dioxide from the recycled off-gas 149. The adsorbed carbon dioxide can be vented from the VPSA system 170 via a vent valve 172 or optionally can be compressed in a low-pressure compressor 175 and directed to a liquefier 180 that produces liquid carbon dioxide 182 for external sale or re-use within the plant as well as a non-condensable waste stream 184.

A variant of the in-line carbon dioxide stripping embodiment would replace the separate oxygen containing gas source and inert carrier gas source with a single source of air. This alternate arrangement would be a closed loop system with the oxygen concentration in the micro-aeration gas mixture sent to the fermentation vessel or reactor starting at about 21% and diminishing thereafter as oxygen is consumed in the fermentation process. Since the recycled off-gas is stripped of carbon dioxide and other unwanted contaminants, the cleansed or purified off-gas becomes oxygen depleted air. This process continues until the oxygen concentration in the off-gas is too low to provide any micro-aeration benefits or the superficial velocity of the micro-aeration gas is too low, when supplemental or make-up air is added to the recycle loop.

A further variant of the above-described embodiments contemplates the use of nutrient containing carrier gases in lieu of or together with the inert carrier gas, for example, in the production of bio-succinic acid. In situations where nutrient containing carrier gases are used, the oxygen containing gas is preferably pure oxygen rather than air. The volume of the nutrient containing carrier gas to be used with the present embodiments would be sufficiently high in order to achieve the desired bulk mixing and improved dispersion of both the oxygen as well as the nutrient containing carrier gas throughout the fermentation broth.

### INDUSTRIAL APPLICABILITY

In one aspect, the present micro-aeration based fermentation system and method may be characterized as a conversion of standard anaerobic fermenters, typically large empty vessels, into a vigorously mixed bubbling column, which would allow the fermentation vessel or reactor to facilitate the micro-aeration and at the same time provide an efficient means for homogeneous mixing of the contents of the fermentation vessel. The homogeneous mixing allows uniform dispersion of the nutrients and dissolved oxygen to the microbes so that every section of the fermentation vessel receives sufficient oxygen to ensure no premature microbe deaths and allowing for optimized fermentation yields. The excess carrier gas provides the necessary flow and motive force necessary to provide an economic means of agitating or mixing the contents of the entire vessel without the need of a mechanical agitator. To create the heterogeneous flow bubbling column necessary for proper mixing, a minimum superficial gas velocity of about 0.02 m/sec is needed, and more preferably the superficial gas velocity should be about 0.05 m/sec or higher.

Another aspect or feature of the present micro-aeration based fermentation system and method is the advanced control system that facilitates improved control of the amount of oxygen being consumed by the microbes, allowing for proper and commensurate scale-up from the laboratory scale fermenters to the larger commercial scale fermenters. With the dissolved oxygen and fermentation broth thoroughly mixed throughout the fermenter, sampling of the fermentation broth is much more representative of the entire batch. This embodiment with the associated advanced control system also allows for the use of off-gas analysis using an oxygen and carbon dioxide analyzer for measuring an actual oxygen uptake rate so that proper scale-up can be achieved based on empirical laboratory results.

Yet another aspect or feature of the present micro-aeration based fermentation system and method is the stripping of unwanted volatile products or byproducts from the fermentation broth. Accumulated dissolved carbon dioxide potentially hinders the metabolic rate of some microbes. For instance, this unwanted carbon dioxide may reduce the tolerance level of yeast to the toxic concentration levels of ethanol, thus reducing peak production rates and/or yields of the desired end products. None of the prior art references suggest a multi-purpose carrier gas arrangement, namely micro-aeration of the fermentation broth; bulk mixing of the fermentation broth; and stripping out the undesirable volatile byproducts or toxic products from the fermentation broth as the fermentation process proceeds. In the present embodiments, the stripping rate is preferably controlled by adjusting the volume and flow rate of the inert carrier gas purging off the undesirable volatiles, as required or stripping unwanted carbon dioxide from the recirculating flow of off-gases.

Another key feature or aspect of the present micro-aeration based fermentation system and method is off-gas recycling in a recirculation or recycle loop. Because of the large volume of carrier gas utilized, recycling the gas in a closed loop system such that the off-gas can be recompressed utilizing blowers and compressors. Oxygen or air is added into the off-gas recycle loop to make-up the depleted oxygen or air necessary to maintain the optimal oxygen concentration within the micro-aeration gas and the resulting DO levels, OUR, and OTR. In addition, supplemental nitrogen gas or other inert carrier gases are added to the off-gas recycle loop to maintain the excess volume and minimum superficial velocity of the micro-aeration gas, as a portion of the off-gas may be purged depending on the need to strip out undesirable volatiles or accumulated carbon dioxide.

By allowing easy scale up and uniform dispersion of the proper amount of oxygen for a micro-aeration based fermentation process, the yield from an optimized anaerobic fermentation process would be higher, resulting in significant additional revenue for the processor. Furthermore, adding the vigorous bulk mixing and stripping to remove undesirable volatiles from the fermentation broth will further increase the product yields and rate of fermentation. With a shorter fermentation batch time, the net cost of production is also greatly reduced. Therefore, the economic benefits of additional yields and lower operating costs will outpace the costs associated with the process changes and capital costs required to adopt the present micro-aeration system.

To make the micro-aeration system and process more economically attractive, the inert carrier gas is preferably recirculated thereby substantially reducing the costs associated with the inert carrier gas compared to many prior art ‘once through’ gas systems and processes. Gas purge/vent systems are used to avoid the buildup of undesirable volatiles such as carbon dioxide and gas makeup delivery are employed to replace such purged or vented gas volume. Although the recirculating gas volume is very large, the volume of the purged gas and corresponding makeup gas is small. The additional yield achieved from the use of the present micro-aeration system and process should more than compensate for costs associated with excessive inert carrier gas usage.

While the present invention has been characterized in various ways and described in relation to preferred embodiments, as will occur to those skilled in the art, numerous, additions, changes and modifications thereto can be made without departing from the spirit and scope of the present invention as set forth in the appended claims.

What is claimed is:

1. A micro-aeration based fermentation system comprising:
a fermentation reactor;
a sparging apparatus disposed in the fermentation reactor; and
a micro-aeration gas mixture delivered to the fermentation reactor via the sparging apparatus, the micro-aeration gas mixture comprising an oxygen containing gas and an inert carrier gas;
an off-gas recycle loop configured to recycle off-gases exiting the fermentation reactor back to the sparger apparatus;
wherein the total oxygen concentration in the micro-aeration gas mixture is less than or equal to 20%, and wherein the micro-aeration gas mixture is delivered to the fermentation reactor at a minimum superficial velocity of 0.02 m/sec to mix the fermentation broth within the fermentation reactor and disperse the oxygen containing gas throughout the fermentation broth.

2. The system of claim 1 wherein said inert carrier gas comprises nitrogen.

3. The system of claim 1 wherein said fermentation reactor has a height to diameter ratio of at least about 3 to 1.

4. The system of claim 1 wherein the sparging apparatus is disposed proximate the bottom of the fermentation reactor and the fermentation reactor is configured without mechanical agitators.

5. The system of claim 1 further comprising a microprocessor based programmable logic controller (PLC) operatively connected to one or more control valves for regulating the flow of the inert carrier gas from a source of inert carrier gas to the fermentation reactor and for regulating the flow of the oxygen containing gas from a source of oxygen containing gas to the fermentation reactor.

6. The system of claim 5 further comprising a plurality of measurement devices operatively connected to the microprocessor based programmable logic controller (PLC) and configured to measure one or more of the gas flows within the micro-aeration based fermentation system and to ascertain physical and chemical characteristics of the fermentation broth and off-gases from the fermentation broth.

7. The system of claim 6 wherein the plurality of measurement devices are selected from the group consisting of flow meters, dissolved oxygen probes, cell density meters, and gas analyzers.

8. The system of claim 6 wherein the volume of the oxygen containing gas and inert carrier gas delivered to the fermentation reactor are controlled or regulated in response to a respiratory quotient (RQ); an oxygen transfer rate (OTR), an oxygen uptake rate (OUR), an carbon dioxide evolution rate (CER) or a combination thereof associated with the fermentation broth.

9. The system of claim 6 further comprising a purge or vent line with one or more control valves disposed therein coupled to the fermentation reactor, the one or more control valves operatively connected to the PLC for regulating the purge or vent of off-gases from the fermentation reactor in response to measured gas concentrations in the off-gas recycle loop so as to reduce the amount of carbon dioxide and other unwanted volatiles recycled back to the sparger apparatus.

10. The system of claim 1 further comprising a blower or gas compressor disposed in the recycle loop and configured to forcibly recirculate the off-gases exiting the fermentation reactor back to the sparger apparatus via the recycle loop.

11. The system of claim 1 further comprising a carbon dioxide stripping subsystem disposed in operative association with the off-gas recycle loop, the carbon dioxide stripping subsystem comprising a variable pressure swing adsorption system configured to adsorb carbon dioxide from the recycled off-gas.

12. The system of claim 11 further comprising a vent operatively coupled to the variable pressure swing adsorption system to vent the carbon dioxide extracted from the recycle loop.

13. The system of claim 11 further comprising a compressor and a liquefier operatively coupled to the variable pressure swing adsorption system to liquefy the carbon dioxide extracted from the recycle loop.

14. A method of micro-aeration of a fermentation broth comprising the steps of:
(i) mixing an inert carrier gas flow with an oxygen containing gas flow and an off-gas recycle flow to produce a micro-aeration gas mixture having an oxygen concentration of less than about 20% by volume;
(ii) sparging the micro-aeration gas mixture to the fermentation broth in a fermentation reactor via a sparging apparatus at a minimum superficial velocity of 0.02 m/sec to mix the fermentation broth within the fermentation reactor and uniformly disperse the oxygen containing gas throughout the fermentation broth;
(iii) recycling some or all of the off-gases produced by the fermentation broth in the fermentation reactor back to the sparger apparatus via an off-gas recycle loop;
(iv) measuring one or more of the gas flows selected from the group consisting of the off-gas recycling flow, the inert carrier gas flow, the oxygen containing gas flow, and the micro-aeration gas flow;
(v) ascertaining selected parameters representing the physical and chemical characteristics of the fermentation broth in the fermentation reactor and selected gas concentrations in the off-gas recycle loop; and
(vi) controlling the flows of the inert carrier gas and the oxygen containing gas in response to the selected parameters.

15. The method of claim 14 wherein said inert carrier gas comprises nitrogen.

16. The method of claim 14 wherein the selected parameters representing the physical and chemical characteristics of the fermentation broth comprise a respiratory quotient (RQ); an oxygen transfer rate (OTR), an oxygen uptake rate (OUR), an carbon dioxide evolution rate (CER) or a combination thereof.

17. The method of claim 14 further comprising the step of purging or venting the off-gases from the fermentation reactor in response to the ascertained gas concentrations in the off-gas recycle loop so as to reduce the amount of carbon dioxide and other unwanted volatiles recycled back to the sparger apparatus.

18. The method of claim 14 further comprising the step of stripping carbon dioxide from the off-gases in the off-gas recycle loop using a variable pressure swing adsorption system configured to adsorb carbon dioxide from the recycled off-gas.

19. The method of claim 18 further comprising the step of venting the carbon dioxide extracted from the recycle loop.

20. The method of claim 18 further comprising the step of liquefying the carbon dioxide extracted from the recycle loop.
